(19) United States
(12) Patent Application Publication
(10) Pub. No.: US 2010/0312058 A1
(43) Pub. Date: Dec. 9, 2010

(54) ENDOSCOPIC APPARATUS AND ENDOSCOPIC IMAGE OBTAINMENT METHOD

(75) Inventors: Masahiro Kubo, Ashigarakami-gun (JP); Kazuo Okoyama, Ashigarakami-gun (JP); Yasunori Ohta, Ashigarakami-gun (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: FUJIFILM Corporation, Tokyo (JP)

(21) Appl. No.: 12/792,605

(22) Filed: Jun. 2, 2010

(30) Foreign Application Priority Data
Jun. 4, 2009 (JP) 134680/2009

(51) Int. Cl. A61B 1/06

(52) U.S. Cl. 600/178

(57) ABSTRACT

When a wavelength set for a spectral estimation image is set through an input means, ON/OFF of light emitting devices is controlled based on the set wavelength set. Further, an endoscopic image of a subject illuminated with light of a predetermined wavelength band or bands is obtained by a scope. Matrix operation is performed on the endoscopic image to generate the spectral estimation image.

### BACKGROUND OF THE INVENTION

1. Field of the Invention

The present invention relates to an endoscopic apparatus that obtain spectral estimation images by matrix operation and an endoscopic image obtainment method.

2. Description of the Related Art

Endoscopic apparatuses are used to diagnose the conditions of the body cavities of patients, such as esophagi, stomachs, and large intestines, based on endoscopic images of the patients. The endoscopic apparatuses obtain the images of the body cavities of patients by inserting scopes into the body cavities. In recent years, when diagnosis is performed by using an endoscope, endoscopic images obtained by a scope are displayed on a monitor in real time. Therefore, a doctor checks imaged positions or regions by looking at the monitor, and diagnoses the patient based on the images (image diagnosis). Further, observation by endoscopes includes an ordinary observation mode, a fluorescent observation mode, a narrow-band mode, and the like to facilitate image diagnosis by doctors. The ordinary observation mode observes the subject illuminated with white light. The fluorescent observation mode observes fluorescence output from the subject when the subject is illuminated with fluorescence. The narrow-band mode observes the subject illuminated with narrow band light.

Further, as light sources for endoscopes, use of LED's (light emitting diodes) instead of tungsten or the like has been proposed (for example, please refer to Japanese Unexamined Patent Publication No. 2006-166940 (Patent Document 1)). An endoscopic apparatus disclosed in Patent Document 1 includes four LED light sources that have different wavelength bands from each other. Further, Patent Document 1 discloses the feature that the LED's and control operations are switched based on an ordinary observation mode, a fluorescent observation mode, and an NBI mode.

Further, generation of spectral images by operation processing based on image signals obtained by using white light has been proposed. The white light is used instead of narrow band light, which is used in a narrow band mode. In this method, relationships between numerical data representing the color sensitivity characteristic of each of R, G and B and numerical data representing the spectral characteristic of a specific narrow band-pass filter are obtained as matrix data (coefficient sets). A spectral estimation image is obtained by estimating a spectral image that will be obtained through the narrow band-pass filter by performing operation using the matrix data and RGB signals. When the spectral image is generated by performing operation as described above, it is not necessary to prepare a plurality of filters corresponding to desirable wavelength bands. Further, it is not necessary to change or arrange the plurality of filters. Therefore, it is possible to prevent the size of the endoscopic apparatus from becoming large, and to lower the cost of the endoscopic apparatus.

Further, when spectral images are generated by matrix operation, changing the intensity of light output from an LED light source has been proposed (for example, please refer to Japanese Unexamined Patent Publication No. 2007-264537 (Patent Document 2)). Specifically, three LED's that have different wavelength bands from each other are prepared. When the wavelength band of a spectral image is selected, the intensity of light of each of the three LED's is changed, and a frame image is obtained for the wavelength band of each of the LED's. Further, a single spectral image is generated by using the three frame images.

However, when a spectral image is generated by using a plurality of frame images, as proposed in Patent Document 2, a time period corresponding to the plurality of frame images is required, and the time period for generating the spectral image becomes long. Therefore, the frame rate of video images (motion images, dynamic images or the like) becomes lower, and the image quality becomes lower. Hence, efficient image diagnosis becomes impossible.

### SUMMARY OF THE INVENTION

In view of the foregoing circumstances, it is an object of the present invention to provide an endoscopic apparatus that can efficiently generate accurate spectral images, and an endoscopic image obtainment method.

An endoscopic apparatus of the present invention is an endoscopic apparatus comprising:

a light source unit including a plurality of light emitting devices that output light of different wavelength bands from each other, the light source unit being able to output white light by driving the plurality of light emitting devices;

a scope that obtains an endoscopic image by imaging a subject illuminated with light output from the light source unit;

a wavelength set setting means that sets a plurality of wavelength bands, as a wavelength set;

a light source control means that controls, based on the wavelength set that has been set by the wavelength set setting means, ON/OFF of each of the plurality of light emitting devices; and

a spectral image generation means that generates a spectral estimation image by performing, based on the wavelength set that has been set by the wavelength set setting means, matrix operation on the endoscopic image by using matrix parameter.

An endoscopic image obtainment method of the present invention is an endoscopic image obtainment method comprising the steps of:

setting a plurality of wavelength bands, as a wavelength set;

controlling a light source unit including a plurality of light emitting devices that output light of different wavelength bands from each other, the light source unit being able to output white light by driving the plurality of light emitting devices, by controlling ON/OFF of each of the plurality of light emitting devices based on the set wavelength set; and

generating a spectral estimation image by performing, based on the set wavelength set, matrix operation on an endoscopic image that has been obtained by a scope while ON/OFF of each of the plurality of light emitting devices is controlled.

Here, the light source control means should control, based on the wavelength set, ON/OFF of the plurality of light emitting devices. When each of the wavelength bands included in the wavelength set overlaps with at least one of the wavelength bands of the plurality of light emitting devices, the light source control means may drive only the light emitting devices of the overlapping wavelength bands. When at least one of the wavelength bands included in the wavelength set overlaps with none of the wavelength bands of the plurality of light emitting devices, the light source control means may drive the plurality of light emitting devices of wavelength bands that are necessary to generate the spectral estimation image.

The method for setting the wavelength set (a plurality of wavelength bands) by the wavelength set setting means is not limited as long as a plurality of wavelength bands to be represented by a spectral estimation image are set. For example, a plurality of wavelengths may be set based on an input by a user. Alternatively, the wavelength set setting means may set the wavelength set by selecting the wavelength set from a plurality of wavelength sets that have been prepared in advance. Further, the light source unit may include the plurality of light emitting devices corresponding to respective wavelength bands included in the plurality of wavelength sets.

Further, when at least one of the wavelength bands included in the wavelength set overlaps with none of the wavelength bands of the plurality of light emitting devices, the light source control means may drive all of the plurality of light emitting devices. Alternatively, the light source control means may drive a combination of light emitting devices selected from the plurality of light emitting devices, the combination achieving highest estimation accuracy.

According to the endoscopic apparatus and the endoscopic image obtainment method of the present invention, a plurality of wavelength bands to be represented by a spectral estimation image are set as a wavelength set. Further, a light source unit including a plurality of light emitting devices that output light of different wavelength bands from each other, the light source unit being able to output white light by driving the plurality of light emitting devices, is controlled. The light source unit is controlled by controlling ON/OFF of each of the plurality of light emitting devices based on the set wavelength set. Further, a spectral estimation image is generated by performing, based on the set wavelength set, matrix operation on an endoscopic image that has been obtained by a scope while ON/OFF of each of the plurality of light emitting devices is controlled. Therefore, it is possible to improve the estimation accuracy of the spectral estimation image by outputting light of a wavelength band or bands that are necessary to generate the spectral estimation image from an endoscopic image. Further, it is possible to efficiently generate a spectral estimation image.

When the wavelength set setting means sets the wavelength set by selecting a wavelength set from a plurality of wavelength sets that have been prepared advance, and the light source unit includes a plurality of light emitting devices corresponding to respective wavelength bands included in the plurality of wavelength sets, if a user selects an arbitrary wavelength set, the light emitting devices are automatically drive-controlled based on the selection by the user. Therefore, it is possible to improve the estimation accuracy of the spectral estimation image and to efficiently generate a spectral estimation image.

Further, when at least one of the wavelength bands included in the wavelength set overlaps with none of the wavelength bands of the plurality of light emitting devices, the light source control means may drive a combination of light emitting devices selected from the plurality of light emitting devices, the combination achieving highest estimation accuracy. When the light source control means operates in such a manner, even if at least one of the wavelength bands included in the wavelength set overlaps with none of the wavelength bands of the plurality of light emitting devices, it is possible to maintain the estimation accuracy of the spectral estimation image at a high level.

### BRIEF DESCRIPTION OF THE DRAWINGS

FIG. 1 is a block diagram illustrating an endoscopic apparatus according to an embodiment of the present invention;

FIG. 2 is a table showing an example of matrix parameters used by a spectral image generation means illustrated in FIG. 1;

FIG. 3 is a table showing an example of corresponding relationships between wavelength sets that are set by a wavelength set setting means illustrated in FIG. 1 and ON/OFF control of each light emitting device; and

FIG. 4 is a flow chart illustrating an endoscopic image obtainment method according to an embodiment of the present invention.

### DESCRIPTION OF THE PREFERRED EMBODIMENTS

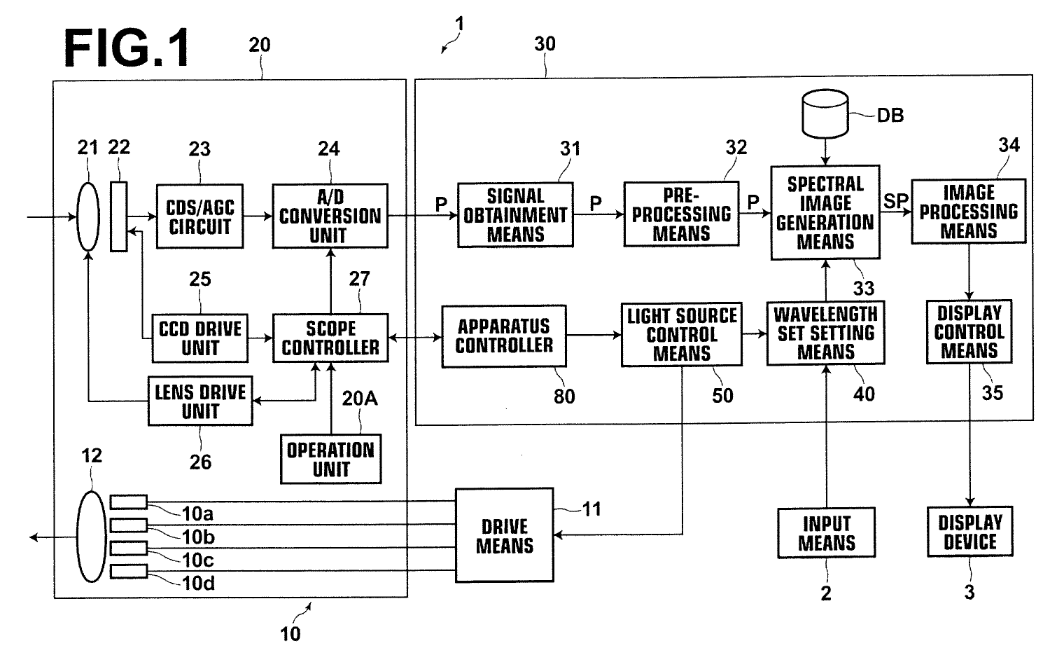

Hereinafter, embodiments of the present invention will be described in detail with reference to drawings. FIG. 1 is a block diagram illustrating an example of an endoscopic apparatus according to the present invention. An endoscopic apparatus 1 includes a light source unit 10, a scope 20, and an endoscopic image processing apparatus 30. The light source unit 10 outputs light to a subject to observe the subject by an endoscope. The light source unit 10 includes a plurality of light emitting devices 10a through 10d that output light of different wavelength bands from each other. The plurality of light emitting devices 10a through 10d are arranged at the leading end of the scope 20. Specifically, the light emitting devices 10a through 10d include LED's the peak wavelengths of which are 470 nm, 500 nm, 540 nm, and 620 nm, respectively. Therefore, white light is output from the light source unit 10 when all of the light emitting devices 10a through 10d are driven or three light emitting devices, namely light emitting devices 10a, 10b (or 10c), and 10d, are driven. The plurality of light emitting devices 10a through 10d emit light by application of drive currents by a drive means 11. Further, the operation of the drive means 11 is controlled by a light source control means 50.

The scope 20 includes an imaging lens 21, an imaging device 22, a CDS/AGC circuit 23, an A/D converter 24, a CCD drive unit 25, a lens drive unit 26, and the like. Each element of the scope 20 is controlled by a scope controller 27. The imaging lens 21 is composed of a plurality of lens groups for example. The imaging magnification of the imaging lens 21 is changed by being driven by a lens drive unit 26. The imaging device 22 includes, for example, a CCD, a CMOS, or the like. The imaging device 22 obtains an image by performing photoelectric conversion on an image of the subject faulted by the imaging lens 21. As the imaging device 22, a complementary color type device, or a primary color type device is used for example. The complementary color type device has a color filter of Mg (magenta), Ye (yellow), Cy (cyan), and G (green) on the imaging surface thereof. The primary color type device has a color filter of RGB. Further, the operation of the imaging device 22 is controlled by a CCD drive unit 25. When the imaging device 22 obtains image (video) signals, the CDS/AGC (correlated double sampling/automatic gain control) circuit 23 performs sampling on the image (video) signals, and amplifies the sampled signals. Further, the A/D converter 24 performs A/D conversion on an endoscopic image output from the CDS/AGC circuit 23, and outputs the converted endoscopic image to the endoscopic image processing apparatus 30.

The endoscopic image processing apparatus 30 processes endoscopic image P, obtained by using the scope 20. For example, the endoscopic image processing apparatus 30 is configured by a DSP (digital signal processor) or the like. The endoscopic image processing apparatus 30 includes an image obtainment means 31, a pre-processing means 32, a spectral image generation means 33, an image processing means, and a display control means 35. The image obtainment means 31 obtains endoscopic image P imaged by the imaging device 22 in the scope 20. The pre-processing means 32 performs pre-processing on the endoscopic image P obtained by the image obtainment means 31. For example, when the endoscopic image P is represented by a YCC color system, the pre-processing means 32 converts the endoscopic image P into an image represented by an RGB color system. Further, the pre-processing means 32 has a gamma conversion function (gamma correction function), a gradation adjustment function, and the like.

The spectral image generation means 33 generates spectral estimation image SP by performing matrix operation on the endoscopic image P by using matrix parameter M. An example of the operation by the spectral image generation means 33 is described in detail in Japanese Unexamined Patent Publication No. 2003-093336.

Specifically, the spectral image generation means 33 generates the spectral estimation image SP by performing matrix operation using the following formula (1):

$$[ Formula 1 ]$$

$$( \begin{matrix}{SP}_{r} \\ {SP}_{g} \\ {SP}_{b}\end{matrix} ) = ( \begin{matrix}{M}_{00} & {M}_{01} & {M}_{02} \\ {M}_{10} & {M}_{11} & {M}_{12} \\ {M}_{20} & {M}_{21} & {M}_{22}\end{matrix} ) \cdot ( \begin{matrix}Pr \\ Pg \\ Pb\end{matrix} ) . \qquad ( 1 )$$

In Formula (1), Spᵣ, Sp_{g}, Sp_{b} represent R, G and B components of the spectral estimation image SP, respectively. Pr, Pg and Pb represent R, G and B components of the endoscopic image P, respectively. Values M₀₀ through M₂₂ in the matrix of 3×3 (three columns and three rows) represent matrix parameters M for matrix operation, respectively.

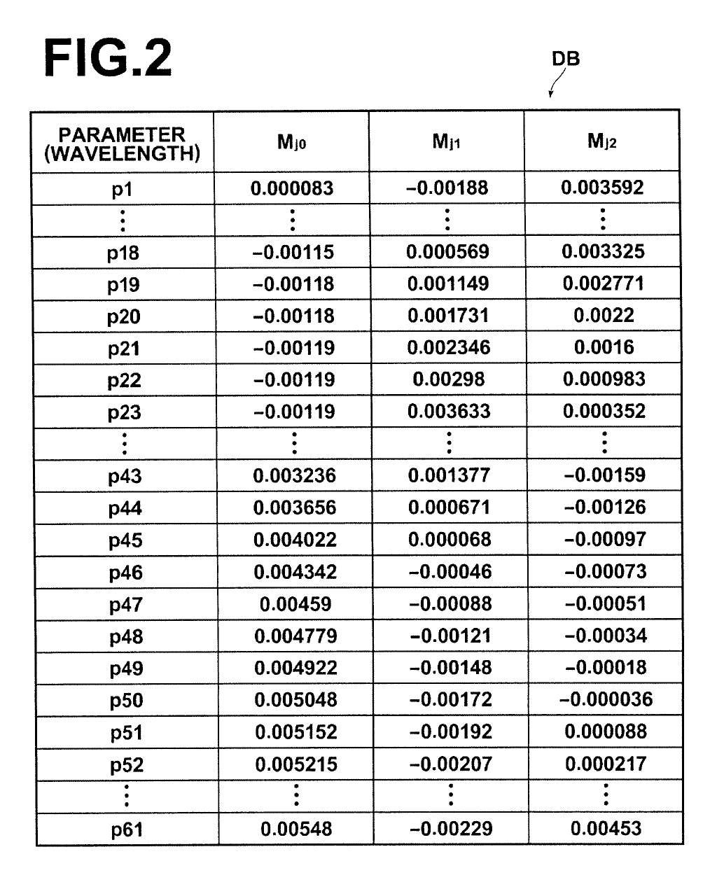

FIG. 2 is a table showing an example of database DB that stores matrix parameters for performing the matrix operation represented by Formula (I). In FIG. 2, the database DB stores parameter pi=(Mⱼ₀, Mⱼ₁, Mⱼ₂) (i is a sign for distinguishing parameter sets stored in the database DB from each other, and i=1 through 61, and j represents the rows of matrix M in Formula (1), and j=0 through 2) for each of 61 wavelength bands, into which the wavelength band of 400 nm to 700 nm is divided at 5 nm intervals.

The wavelength set setting means 40 illustrated in FIG. 1 sets, as a wavelength set, a plurality of wavelength bands to be represented by spectral estimation image SP. For example, the wavelength set setting means 40 has, in advance, a plurality of wavelength sets that are appropriate to observe respective regions, such as blood vessels and a living body tissue. The wavelength set setting means 40 sets a wavelength set by selecting the wavelength set based on an input from an input means. Specifically, for example, three wavelength sets CH1 (550 nm, 500 nm, 470 nm), CH2 (525 nm, 495 nm, 495 nm), and CH3 (540 nm, 415 nm, 415 nm) are prepared as wavelength sets for setting matrix parameter M. Further, the wavelength set setting means 40 may have a function for setting, as the wavelength set for spectral estimation image SP, an arbitrary wavelength set based on an input by a user, instead of the wavelength set that has been prepared in advance.

An image processing means 34 illustrated in FIG. 1 performs enhancement processing or the like on endoscopic image p and spectral estimation image SP. A display control means 35 has a function for displaying the endoscopic image P and the spectral estimation image SP on which image processing has been performed by the image processing means 34. The display control means 35 displays the images on a display device 3 together with character information or the like.

A light source control means 50 has a function for controlling the light emitting devices 10a through 10d based on the wavelength set that has been set by the wavelength set setting means 40. Specifically, first, the light source control means 50 judges whether each of all the wavelength bands included in the wavelength set that has been set by the wavelength set setting means 40 overlaps with (or is substantially the same as) at least one of the wavelength bands of the light emitting devices 10a through 10d.

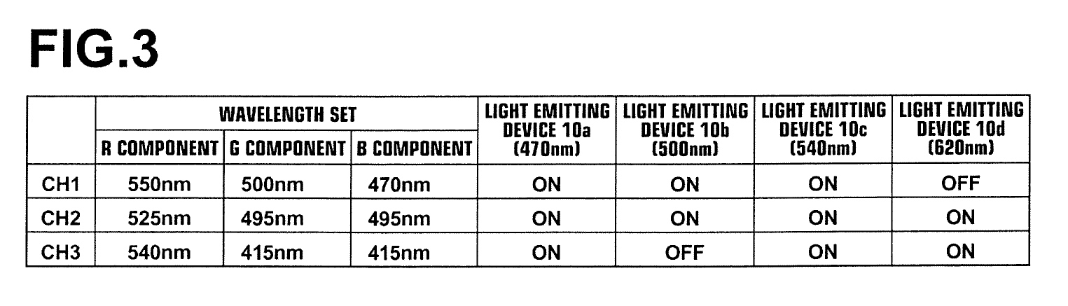

When the light source control means 50 judges that each of all the wavelength bands included in the wavelength set that has been set by the wavelength set setting means 40 overlaps with at least one of the wavelength bands of the light emitting devices 10a through 10d, the light source control means 50 drives only the light emitting device or devices the wavelength band or bands of which overlap. For example, as illustrated in FIG. 3, when wavelength set CH1 including, as components of spectral estimation image SP, R component of 550 nm, G component of 500 nm, and B component of 470 nm is set, the wavelength bands included in the wavelength set are substantially the same as the wavelength bands of the light emitting devices 10a, 10b and 10c of the light emitting devices 10a through 10d. Therefore, the light source control means 50 controls the light emitting device 10d to OFF state, and the light emitting devices 10a, 10b, and 10c to ON state so that the endoscopic image P is obtained while the light emitting devices 10a, 10b and 10c are emitting light.

In contrast, when at least one of the wavelength bands included in the wavelength set overlaps with none of the wavelength bands of the light emitting devices, the light source control means 50 drives the light emitting devices 10a through 10d of wavelength bands that are necessary to generate the spectral estimation image SP. Specifically, when the wavelength bands included in the wavelength set overlap with none of the wavelength bands of the light emitting devices 10a through 10d, as in wavelength set CH2 in FIG. 3, the light source control means 50 drives all of the light emitting devices 10a through 10d to illuminate the subject with white light. Further, the spectral image generation means 33 generates, by using Formula (1), spectral estimation image SP from the endoscopic image P that has been obtained by illumination with white light.

Alternatively, the light source control means 50 may drive a combination of light emitting devices selected from the plurality of light emitting devices, the combination achieving the highest estimation accuracy. Specifically, the light source control means 50 stores, in advance, a combination of ON/OFF of the light emitting devices 10a through 10d, the combination achieving the highest spectral estimation accuracy when an observation region corresponding to each wavelength set, or a color patch that has substantially the same color as the observation region is illuminated with light by combining ON/OFF of each of the light emitting devices 10a through 10d. For example, when wavelength bands of a wavelength set overlap with a part of the wavelength bands of the light emitting devices 10a through 10d, as in wavelength set CH3 illustrated in FIG. 3, the light source control means 50 controls ON/OFF of the light emitting devices 10a through 10d by using a combination of ON/OFF of each of the light emitting devices 10a through 10d that has been stored in advance.

As described above, the wavelength bands of light output from the light source unit 10 are selected based on the set wavelength sets CH1 through CH3. Therefore, it is possible to efficiently generate spectral estimation image SP that has high estimation accuracy. As in a conventional method, when a spectral image is obtained by using an optical filter, the structure of the apparatus tends to become complex. Further, when images are obtained, as separate frame images, by illumination with light having different wavelength bands from each other, and matrix operation is performed, the frame rate becomes lower, and efficient image diagnosis is impossible. In contrast, in the present invention, the wavelength bands of light output from the light source unit 10 are selected based on the wavelength sets CH1 through CH3, and the light source unit 10 is driven to obtain a single endoscopic image P by illumination with light of the selected wavelength bands. Further, spectral estimation image SP is obtained by performing matrix operation on the single endoscopic image P. Therefore, it is possible to generate the spectral estimation image SP from the endoscopic image P, which corresponds to one frame image, in simple apparatus structure that does not require an optical filter and the like. Hence, efficient image diagnosis is possible.

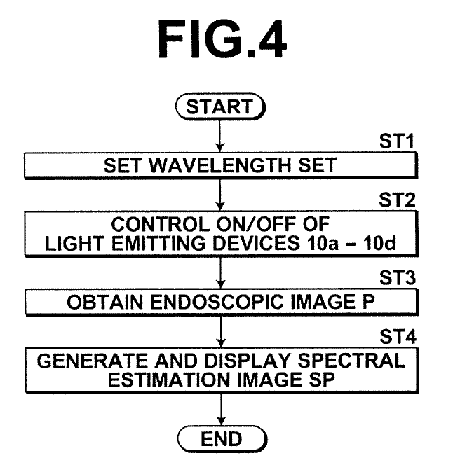

FIG. 4 is a flow chart illustrating an endoscopic image obtainment method according to an embodiment of the present invention. The endoscopic image obtainment method will be described with reference to FIGS. 1 through 4. First, imaging is performed by the scope 20 inserted into the body cavity of a patient. At this time, the user selects one of wavelength sets CH1 through CH3 by using an input means 2, and the wavelength set setting means 40 sets the wavelength set for spectral estimation image SP (step ST1).

Further, the light source control means 50 drives the plurality of light emitting devices 10a through 10d by controlling ON/OFF of the plurality of light emitting devices 10a through 10d based on the wavelength set that has been set by the wavelength set setting means 40 (step ST2, please refer to FIG. 3). Further, the scope 20 images subject S illuminated with light of a predetermined wavelength band or bands to obtain endoscopic image P (step ST3). After then, the spectral image generation means 33 performs matrix operation on the endoscopic image P by using Formula (1) to generate spectral estimation image SP. Further, the display device 3 displays the spectral estimation image SP (step ST4).

According to the embodiments of the present invention, a plurality of wavelength bands to be represented by spectral estimation image SP are set as wavelength sets CH1 through CH3. Further, the light source unit 10 including the plurality of light emitting devices 10a through 10d, which output light of different wavelength bands from each other, is controlled. The light source unit 10 can output white light by driving the plurality of light emitting devices 10a through 10d. ON/OFF of each of the plurality of light emitting devices 10a through 10d is controlled based on the set wavelength set CH1 through CH3. Further, endoscopic image P is obtained by imaging the subject by the scope 20 while ON/OFF of each of the light emitting devices 10a through 10d is controlled. Further, matrix operation based on wavelength set CH1 through CH3 is performed on the endoscopic image P obtained by the scope 20 to generate spectral estimation image SP. Therefore, in the present invention, light of wavelength bands that are necessary to generate the spectral estimation image SP from the single endoscopic image P is output. Hence, the present invention can improve the estimation accuracy of the spectral estimation image SP, and efficiently generate the spectral estimation image SP.

Further, when the wavelength set setting means 40 sets a wavelength set by selecting one of the plurality of wavelength sets CH1 through CH3, which have been prepared in advance, and the light source unit 10 includes a plurality of light emitting devices corresponding to respective wavelength bands included in the plurality of wavelength sets CH1 through CH3, if the user selects an arbitrary wavelength set, the user can automatically drive-control the light emitting devices 10a through 10d based on the selected wavelength set. Therefore, it is possible to improve the estimation accuracy of the spectral estimation image SP, and to efficiently generate the spectral estimation image SP.

Further, when at least one of the wavelength bands included in the wavelength sets CH1 through CH3 overlaps with none of the wavelength bands of the light emitting devices 10a through 10d, if the light source control means 50 drives a combination of light emitting devices 10a through 10d, the combination achieving highest estimation accuracy, it is possible to maintain estimation accuracy of the spectral estimation image SP at a high level even if at least one of the wavelength bands included in the wavelength sets overlaps with none of the wavelength bands of the light emitting devices 10a through 10d.

The embodiments of the present invention are not limited to the embodiments described above. For example, a case in which the light source unit 10 includes four kinds of light emitting devices (LED) 10a through 10d was described. However, the number of the kinds of light emitting devices is not limited to four, and the number of the kinds may be greater than four. Further, a case in which each of the light emitting devices 10a through 10d is arranged at the leading end of the scope 20 was described as an example. Alternatively, the light emitting devices 10a through 10d may be provided in a separate unit, which is different from the scope 20, and light transmitted through a light guide, such as an optical fiber, may be output to the subject from the leading end of the scope 20.

What is claimed is:

1. An endoscopic apparatus comprising:
a light source unit including a plurality of light emitting devices that output light of different wavelength bands from each other, the light source unit being able to output white light by driving the plurality of light emitting devices;
a scope that obtains an endoscopic image by imaging a subject illuminated with light output from the light source unit;
a wavelength set setting means that sets a plurality of wavelength bands, as a wavelength set,;
a light source control means that controls, based on the wavelength set that has been set by the wavelength set setting means, ON/OFF of each of the plurality of light emitting devices; and
a spectral image generation means that generates a spectral estimation image by performing, based on the wavelength set that has been set by the wavelength set setting means, matrix operation on the endoscopic image by using matrix parameter.

2. An endoscopic apparatus, as defined in claim 1, wherein when each of the wavelength bands included in the wavelength set overlaps with at least one of the wavelength bands of the plurality of light emitting devices, the light source control means drives only the light emitting devices of the overlapping wavelength bands, and wherein when at least one of the wavelength bands included in the wavelength set overlaps with none of the wavelength bands of the plurality of light emitting devices, the light source control means drives the plurality of light emitting devices of wavelength bands that are necessary to generate the spectral estimation image.

3. An endoscopic apparatus, as defined in claim 1, wherein the wavelength set setting means sets the wavelength set by selecting the wavelength set from a plurality of wavelength sets that have been prepared in advance, and wherein the light source unit includes the plurality of light emitting devices corresponding to respective wavelength bands included in the plurality of wavelength sets.

4. An endoscopic apparatus, as defined in claim 3, wherein when at least one of the wavelength bands included in the wavelength set overlaps with none of the wavelength bands of the plurality of light emitting devices, the light source control means drives the plurality of light emitting devices of wavelength bands that are necessary to generate the spectral estimation image.

5. An endoscopic apparatus, as defined in claim 3, wherein when at least one of the wavelength bands included in the wavelength set overlaps with none of the wavelength bands of the plurality of light emitting devices, the light source control means drives the light emitting devices so that the light source unit outputs white light.

6. An endoscopic apparatus, as defined in claim 4, wherein when at least one of the wavelength bands included in the wavelength set overlaps with none of the wavelength bands of the plurality of light emitting devices, the light source control means drives a combination of light emitting devices selected from the plurality of light emitting devices, the combination achieving highest estimation accuracy.

7. An endoscopic image obtainment method comprising the steps of:
setting a plurality of wavelength bands, as a wavelength set;
controlling a light source unit including a plurality of light emitting devices that output light of different wavelength bands from each other, the light source unit being able to output white light by driving the plurality of light emitting devices, by controlling ON/OFF of each of the plurality of light emitting devices based on the set wavelength set; and
generating a spectral estimation image by performing, based on the set wavelength set, matrix operation on an endoscopic image that has been obtained by a scope while ON/OFF of each of the plurality of light emitting devices is controlled.
